(19) United States
(12) Patent Application Publication
(10) Pub. No.: US 2010/0217161 A1
(43) Pub. Date: Aug. 26, 2010

(54) Delivery of therapeutic focused energy

(76) Inventors: Avi Shalgi, Tel Aviv (IL); Avi Mendelson, Hinanit (IL)

Correspondence Address: FENNEMORE CRAIG, 3003 NORTH CENTRAL AVENUE, SUITE 2600, PHOENIX, AZ, 85012, US

(21) Appl. No.: 12/379,565

(22) Filed: Feb. 25, 2009

(51) Int. Cl. A61N 7/00; A61B 6/00

(52) U.S. Cl. 601/2; 600/476; 600/473

(57) ABSTRACT

A system for delivering ultrasonic focused energy, the system comprising a transducer unit for delivering the ultrasonic focused energy, the transducer unit comprising an interface medium adapted to contact at least a portion of a treatment region in a treatment area, and further comprising an electromagnetic (EM) radiating element adapted to transmit EM radiation towards the interface medium, wherein a reflection of the EM radiation from the interface medium is indicative of an extent of acoustic contact; and an energy processing unit adapted to send electrical energy to the transducer unit.

### FIELD

Embodiments of the disclosure relate to an improved delivery of therapeutic focused energy.

### BACKGROUND

Therapeutic focused energy delivery (TFED) is a non-invasive method commonly used in the medical field, both for diagnostic and for therapeutic purposes. An ultrasound scanner is an example of one of the applications of TFED. By delivering a focused ultrasound energy beam to a region of tissue, certain medical conditions may be diagnosed, such as tumors and renal stones. Ultrasound is also used for monitoring fetus development during pregnancy.

TFED may be used for ablation and/or destruction of pathogenic objects and various tissues. TFED may also selectively target and disrupt subcutaneous fat cells and adipose tissue in different parts of a body during non-invasive body contouring procedures. Generally, TFED comprises delivering energy into a confined region in a body. For convenience hereinafter, the confined region may also be referred to as a target area. A transducer element comprised in a transducer unit and adapted to deliver the energy, produces a focused energy beam (TFED beam) whose intensity increases as the beam's cross sectional area decreases towards a focal point in the target area. Intensity may be defined as the energy carried by the TFED beam perpendicularly through unit area per second. At the focal point, the area of the TFED beam is the smallest and therefore intensity is maximal.

A major consideration in TFED is to minimize attenuation in the TFED beam as the beam travels in a path from the transducer element to the target area in the patient's body. Attenuation may be defined as a drop in the intensity of the TFED beam and may be attributed to several factors which include absorption (energy converted into heat), scatter (energy of beam is spread over a wider area), beam divergence (beam diverges and energy is spread over a wider area), reflection (beam is reflected at interfaces between materials along a path of the beam), and refraction (beam is spread at curved interfaces along a path of the beam).

In TFED, acoustic contact is generally associated with, but not limited to, loss of energy due to reflection from air medium. Typically, a beam traveling from the transducer element to the focal point goes through an oil-type fluid adapted to conduct the beam inside the transducer unit; through an interface medium, which may be, for example, a polyethylene membrane, comprised in the transducer unit and adapted to abut with a skin surface in a treatment region; and through a skin surface and other tissue in the treatment area until reaching the target area. A treatment area may be defined as a whole area in the body which will be exposed to TFED over a period of time; a treatment region defined as a region within the treatment area exposed to TFED at a same time. Differences in material composition, for example, between the fluid and the polyethylene membrane, and/or between the membrane and the skin, create mismatches at the interfaces which, depending on the level of mismatch, cause the beam to be partially or wholly reflected. Acoustic contact may be considered to be adequate (adequate acoustic contact) when the level of mismatch does not interfere with sufficient beam energy reaching the target area so as to provide therapeutic results. Acoustic contact may be considered to be inadequate when a degree of mismatch causes a substantial portion of the transmitted energy to be reflected back to the transducer such that insufficient, if any, beam energy reaches the target area to provide therapeutic results. Acoustic contact (AC) may be measured as a ratio of a transmitted and reflected acoustic signal (AC=T/R, wherein for good acoustic contact the AC value will be higher than for bad acoustic contact, since most of the energy is transmitted and not reflected from the medium's interface).

Acoustic contact may be substantially degraded if a layer of air exists between the interface medium and the patient's skin. In such cases, in order to reach the skin, the focused energy beam encounters a mismatch between the interface medium and air, and a mismatch between the air and the skin in the treatment region, generally resulting in a relatively small proportion of the focused energy, if any, reaching the skin. Occasionally, for example but not limited to (may also be oil, etc.), a gel is used as a “matching layer” between the interface medium and the skin, the gel adapted to substantially eliminate the layer of air.

A number of therapeutic focused energy delivery (TFED) apparatuses use acoustic reflection measurement techniques to determine whether adequate acoustic contact exists between a device, such as a transducer unit, and a treatment region in a body. Many of these techniques include the use of an A-mode transducer, generally comprised in the transducer unit, the A-mode transducer adapted to transmit a relatively highly directional, high frequency, short-duration pulse in a direction of the treatment region. The A-mode transducer is further adapted, following transmission, to act as a receiver of acoustic signals reflected back from the interface medium. An intensity (for example, energy, power), amplitude and/or other parameters of the reflected acoustic signals are then compared to a predetermined threshold. If the intensity, amplitude, and/or other comparative parameters, exceed the threshold, an indication is generally given to a treatment provider indicative of there being inadequate acoustic contact between the transducer unit and the treatment region.

### SUMMARY

An aspect of some embodiments relates to providing a method for measuring acoustic contact substantially over a whole treatment region, and to providing a device, and a system, adapted to perform the same. Measuring acoustic contact over substantially the whole treatment region essentially comprises spatially covering a major portion of the interface medium when performing the measurements.

According to an aspect of some embodiments, there is provided a device, which is adapted to spatially cover the interface medium during acoustic contact measurements. Spatially covering the interface medium comprises the use of an electromagnetic (EM) radiating element in the transducer unit, which may be a plurality of radiating elements, adapted to radiate EM radiation which impinges on a major portion of the interface medium at relatively low angles of incidence. The interface medium, which comprises a relatively high index of refraction compared to materials, which generally tend to substantially interfere with acoustic contact, such as for example air, is adapted to substantially totally reflect the impinging EM wave when in contact with the material (air). These materials (air) may be referred to hereinafter, as “non-optical contact” materials. Furthermore, the interface medium is adapted to substantially allow the EM wave to pass through into materials which are acoustically conducting, that is, which have indices of refraction substantially similar to that of the medium, such as for example oil and/or skin. These materials may be referred to hereinafter, as “optical contact” materials. In some embodiments of the invention, the EM radiation may comprise visible light, infrared (IR) light including near infrared light (NIR), ultraviolet (UV) light, laser, or any other type of EM radiation adapted to be totally, or optionally partially, reflected by the interface medium when in contact with a non-optical contact material, and/or further adapted to illuminate the skin adjacent to the optical contact materials. Optionally, a same interface medium may be adapted to illuminate optical contact materials when exposed to one or more different types of EM radiation.

According to an aspect of some embodiments, the EM radiation may pass into an optical contact material in areas of the interface medium, which contact the optical contact material, and may be reflected in other areas of the interface medium which contact a non-optical contact material. A boundary of contact between the interface medium and the contact material may be referred to hereinafter as a refractive boundary. Therefore, an interface medium in contact with both an optical contact material, such as for example, skin, and a non-optical contact material, such as air, will substantially only reflect the EM radiation impinging on areas of the interface medium in contact with air.

According to an aspect of some embodiments, the transducer unit further comprises a detector adapted to reproduce an image, and/or an image map, of the treatment region wherein are shown areas of acoustic contact. Optionally, areas of inadequate acoustic contact are shown. The detector may comprise, for example, an optical imager adapted to capture an image of a treatment area illuminated by light from the radiating elements. Optionally, the detector may comprise a thermal imaging unit, for example, a thermographic imaging device adapted to detect IR in the 8-12 micron range. Optionally, the detector may comprise a camera such as a CCD (charge coupled device) or a CMOS (complementary metal-oxide semiconductor) camera.

In an embodiment, the transducer unit comprises one or more LEDs (light emitting diodes) as the radiating elements. The LEDs are placed at several locations relatively close to the edge of the interface medium, adapted to emit light, which impinges on the interface medium at relatively low angles of incidence. The angles of incidence, which are measured with respect to a normal to the refractive boundary, may be the same or greater than a critical angle. The critical angle is the angle of incidence above which total internal reflection occurs, and may be, for example, 41.8 degrees. Optionally, the critical angle may be greater or lesser than 41.8 degrees, depending on the type of interface medium and/or contact material.

The angle of incidence is measured with respect to the normal at the refractive boundary. Reference is now made to FIG. 1A, which illustrates a critical angle θc, expressed by Snell's Law for an angle of refraction of 90°, and is given by:

θc=arcsin(n2/n1)

where n2 is the refractive index of the less dense medium, and n1 is the refractive index of the denser medium. When the incident ray on the boundary is at the critical angle, the refracted ray is tangent to the boundary at the point of incidence. For example, for visible light traveling from a glass such, as for example, Lucite with an index of refraction of 1.50, into air (index of refraction of 1.00), the critical angle θc may be calculated as follows:

θc=arcsin(1.00/1.50)=41.8°

where n2 is the index of refraction of air (1.00) and n1 is the index of refraction of Lucite (1.50), for light passing from Lucite into air, if the incident angle is at the above critical angle, the refracted beam would be on the border of the glass-air border. If the incident angle is greater than the critical angle, the light would be reflected. For an incident angle lesser than the critical angle, the light would pass from the glass into the air.

In some embodiments, the interface medium, which may be a polyethylene membrane with a refraction index of approximately 1.5, is adapted to allow light to pass through areas of the membrane in contact with skin, and optionally oil. Furthermore, the interface medium is further adapted to reflect light from those areas of the membrane in contact with air (which has a refraction index of approximately 1).

In some embodiments of the invention, the transducer unit comprises a dome-like shaped transducer element adapted to radiate focused energy, for example, ultrasound, towards a focal point in a target area within a treatment region. The transducer element may comprise one or more transducer elements adapted to convert electrical energy into ultrasound. Optionally, the transducer element may be shaped as a flat plate. Optionally, the transducer element may comprise an elongated convex shape. Optionally, the transducer may comprise any shape adapted to focus the focused energy to a focal point in a target area, by direct focusing or optionally, by the use of lenses and/or other radiation focusing devices.

In some embodiments of the invention, the detector comprises a camera with a field-of-view (FOV) adapted to substantially cover the whole area of the polyethylene membrane. Images reproduced by the camera may show areas in the treatment region where there is inadequate acoustic contact as relatively dark areas. This is due to inadequate illumination of the skin (light was reflected from the interface medium and did not reach the optical contact material). Furthermore, areas where there is acoustic contact are shown in the images as illuminated. This is due to light reaching the skin. Optionally, an imaging lens is used in lieu of the camera, the image displayed on an imaging (optical) screen on an opposite side of the lens from that of the interface medium. Optionally, the imaging screen may be liquid crystal display (LCD). Optionally, mirrors may be used to direct the image of the illuminated areas to a remote imaging screen.

In some embodiments of the invention, image processing is used to provide visual indication of an acoustic contact level. A system operator may view images reproduced by the camera and cognitively decide whether there is adequate acoustic contact. Optionally, the system may initiate an alarm if the acoustic contact is below a predetermined threshold, for example, if less than 90% of the treatment region. Optionally, for example, less than 80% of the treatment region, less than 70% of the treatment region, less than 60% of the treatment region, less than 50% of the treatment region. Additionally or alternatively, the system may provide the system operator with interactive visual feedback of the acoustic contact so that corrective measures may be taken by the operator when there is inadequate acoustic contact.

In some embodiments of the invention, the system is adapted to track a position of the transducer unit over a treatment area on a patient's body. The treatment area may be marked with a tracking grid, the tracking grid adapted to divide the treatment area into traceable treatment regions. The treatment regions may comprise squares identified by a roman numeral or a letter. Optionally, the treatment regions may comprise other shapes such as circular, triangular, rectangular, or any other polygonal shape, or any combination thereof, and/or may be optionally identified by symbols, colors, and/or any type of markings, suitable for distinguishing one treatment region from another. A visual indicator such as a crosshair may optionally be marked on the polyethylene membrane, the indicator adapted to correlate the position of the transducer unit with respect to a particular treatment region in the treatment area. An image of the indicator over the tracking grid is reproduced by a detector, for example a camera, the image adapted to provide a system operator with a substantially exact location of the transducer unit over the treatment region. Optionally, the image changes as the operator manually moves the transducer unit, including the indicator, over the tracking grid. Optionally, the transducer unit may be moved over the tracking grid, for example, by a robot arm. Optionally, movement by the robot arm may be operator controlled. Additionally or alternatively, robot arm movement is automatic, guided by comparing the processed images with pre-stored information of the tracking grid. Automatic movement of the robot arm may follow a raster pattern, or optionally a non-raster pattern. Optionally, the automatic movement may be top-bottom, or left-right, clockwise, or counter-clockwise, or any combination thereof.

In some embodiments of the invention, the tracking grid may be painted (or drawn) over the treatment area with visible ink, or optionally invisible ink which is visible only when exposed to EM radiation, for example, light. Optionally, the tracking grid may be preformed and adhered to the treatment area. Optionally, the tracking grid may be prepared over the treatment area using an adhesive material adapted to transfer the TFED without causing substantial interference or reflection. Optionally, the tracking grid may comprise a gel pad which is placed over the treatment area outlining each treatment region.

In some embodiments of the invention, the tracking grid may comprise a matrix code (bar code) wherein each treatment region is identified by a bar code adapted to be read by a bar code reader. The bar code is decoded by the bar code reader and the information extracted may be compared with preprogrammed information in the system in order to determine the position of the transducer. The bar code reader may comprise an electromagnetic scanner such as, for example, a photodiode/light scanner, a laser scanner, an LED scanner, a 2 dimensional (2D) imaging scanner, and the like.

In some embodiments of the invention, the treatment regions may be adapted to provide visual feedback to the operator according to an amount of energy delivered to the region; for example, a color of the region may change, or markings are changed or disappear, and the like. Additionally or alternatively, the visual feedback is provided when the energy delivered has reached a predetermined maximum amount. Optionally, the system is adapted to correlate a position of the transducer unit on the tracking grid with the amount of energy delivered to the treatment region. Optionally, the transducer unit comprises a rolling element, for example a roller, adapted to roll over the tracking grid when the transducer unit is moved from treatment region to treatment region. Movement of the roller is accompanied by the system providing a visual and/or aural feedback, the feedback adapted to alert the operator of transducer unit movement following delivery of focused energy in a treatment region.

There is provided, in accordance with an embodiment, a transducer unit for delivering ultrasonic focused energy, the transducer unit comprising an interface medium adapted to contact at least a portion of a treatment region in a treatment area, and an electromagnetic (EM) radiating element adapted to transmit EM radiation towards the interface medium, wherein the reflection of the EM radiation from the interface medium is indicative of an extent of acoustic contact. Optionally, the EM radiation comprises visible light. Optionally, the EM radiation comprises laser. Optionally, the EM radiation comprises infrared (IR). Optionally, radiation comprises infrared (IR). Optionally, the EM radiation comprises near infrared (NIR) radiation. Optionally, the EM radiation comprises ultraviolet (UV) radiation.

In some embodiments, the EM radiating element is adapted to transmit EM radiation such that the radiation will impinge on the interface medium at a low angle of incidence. Optionally, the low angle of incidence ranges from 35 degrees to 90 degrees. Additionally or alternatively, the EM radiating element is an LED (light emitting diode). Optionally, the interface medium is adapted to reflect at least a portion of the EM radiation in areas of inadequate acoustic contact within the treatment region. Optionally, the interface medium is adapted to pass a portion of the EM radiation in areas of adequate acoustic contact with the treatment region. Additionally or alternatively, the EM radiation spatially cover the interface medium.

In some embodiments, the transducer further comprises a detector adapted to acquire detected EM radiation reflected from the treatment region. Optionally, the detector is further adapted to acquire an image of the treatment region from the detected EM radiation. Optionally, the image comprises areas of acoustic contact. Additionally or alternatively, the image comprises areas of inadequate acoustic contact. Optionally, the detector comprises an optical imager. Optionally, the optical imager comprises a camera.

In some embodiments, the interface medium is a polyethylene membrane. Optionally, the membrane may be any material essentially transparent to EM and/or to acoustic waves, such as, for example, PVC, silicone, and/or the like. Optionally, the interface medium comprises an indicator adapted to show a focal point position of the focused energy.

In some embodiments, the transducer is further adapted for lysing adipose tissue. Optionally, the transducer is further adapted for lysing cellulite.

There is provided, in accordance with an embodiment, a system for delivering ultrasonic focused energy, the system comprising a transducer unit for delivering the ultrasonic focused energy; the transducer unit comprising an interface medium adapted to contact at least a portion of a treatment region in a treatment area, and further comprising an electromagnetic (EM) radiating element adapted to transmit EM radiation towards the interface medium, wherein the reflection of the EM radiation from the interface medium is indicative of an extent of acoustic contact; and an energy processing unit adapted to send electrical energy to the transducer unit. Optionally, the transducer unit further comprises a detector adapted to acquire images of the treatment region.

In some embodiments, the system further comprises a tracking system adapted to process the images acquired by the detector, and to generate tracking images of a position of the transducer unit in the treatment area. Optionally, the transducer unit comprises a indicator adapted to show a focal point position of the focused energy within the treatment region. Optionally, the transducer unit is adapted to acquire an image of the indicator over the treatment region. Optionally, the system further comprises a tracking grid dividing the treatment area into a plurality of treatment regions. Optionally, the tracking grid is painted or drawn on a skin of a patient. Optionally, the tracking grid is adhered to a skin of a patient. Additionally or alternatively, the indicator is adapted to correlate the position of the transducer unit with a treatment region in the tracking grid.

In some embodiments, the tracking grid is adapted to provide visual feedback related to a treatment region which has received a predetermined amount of focused energy. Optionally, the visual feedback is a change of color in the treatment region. Optionally, the visual feedback is an appearance of a marking and/or a symbol in the treatment region. Additionally or alternatively, the visual feedback is a disappearance of a marking and/or a symbol from the treatment region.

There is provided, in accordance with an embodiment, a method for delivering ultrasonic focused energy, the method comprising establishing a contact between an interface medium comprised in a transducer unit for delivering the ultrasonic focused energy, with at least a portion of a treatment region in a treatment area; and transmitting EM radiation the towards the interface medium, wherein the reflection of the EM radiation from the interface medium is indicative of an extent of acoustic contact; and transmitting electrical energy to the transducer unit. Optionally, the method further comprises acquiring images of the treatment region. Optionally, the method further comprises processing the images and generating tracking images of a position of the transducer unit in the treatment area.

In some embodiments, the method further comprises showing a focal point position of the focused energy within the treatment region using a indicator comprised in the transducer unit. Optionally, the method further comprises acquiring an image of the indicator over the treatment region. Additionally or alternatively, the method further comprises correlating the position of the transducer unit with a treatment region in the target grid using the indicator.

In some embodiments, the method further comprises dividing the treatment area into a plurality of treatment regions within a tracking grid. Optionally, the method further comprises painting the tracking grid on a skin of a patient. Optionally, the method further comprises adhering the tracking grid to a skin of a patient.

In some embodiments, the method further providing visual feedback in the tracking grid related to a treatment region which has received a predetermined amount of focused energy. Optionally, the visual feedback is a change of color in the treatment region. Optionally, the visual feedback is an appearance of a marking and/or a symbol in the treatment region. Optionally, the visual feedback is a disappearance of a marking and/or a symbol from the treatment region.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1A schematically shows a critical angle which may be expressed by Snell's Law;

FIG. 1B schematically shows an exemplary prior art acoustic contact measurement system;

FIG. 2A schematically shows an exemplary system for delivering therapeutic focused energy demonstrating full acoustic contact;

FIG. 2B schematically shows an exemplary system for delivering therapeutic focused energy demonstrating no acoustic contact;

FIG. 2C schematically shows an exemplary system for delivering therapeutic focused energy demonstrating partial acoustic contact;

FIG. 3 schematically shows an exemplary treatment tracking grid and a crosshair;

FIG. 4 schematically shows an exemplary system for delivering therapeutic focused energy, comprising an automatic transducer-positioning element;

FIG. 5 schematically shows an exemplary system for delivering therapeutic focused energy, comprising a manual transducer-positioning element;

FIG. 6 schematically shows an exemplary system for delivering therapeutic focused energy comprising an imaging screen in a transducer unit; and

FIGS. 7A-7D show exemplary images of different optical contact materials, as acquired by a TFED system comprising a transducer unit comprising three EM radiating elements (LEDs).

### DETAILED DESCRIPTION

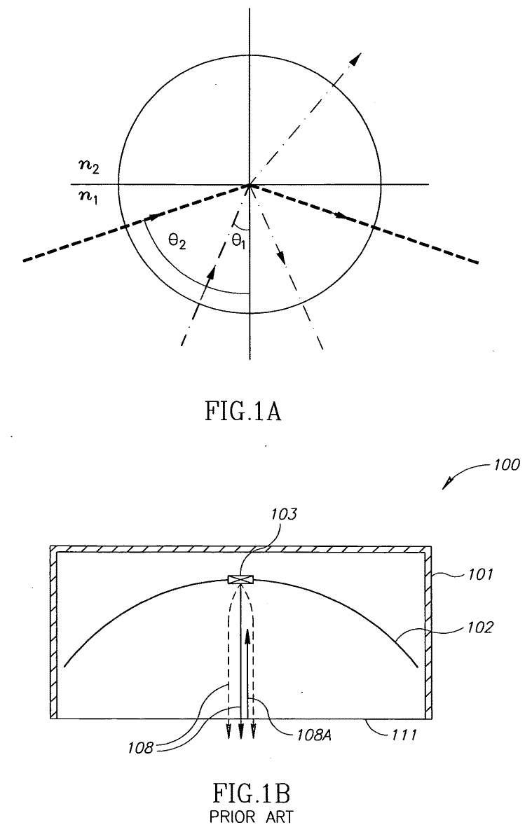

Reference is made to FIG. 1B, which schematically shows an exemplary acoustic contact measurement system 100 as known in the art. Acoustic contact measurement system 100 is adapted to measure acoustic contact between a transducer unit 101 comprised in the measurement system, and a treatment region in a patient (not shown). Transducer unit 101 includes a transducer element 102, an A-mode transducer 103, and an interface medium 111.

A-mode transducer 103 is adapted to transmit a relatively highly directional, high frequency, short-duration pulse 108 through interface medium 111, towards the treatment region. A-mode transducer 103 is further adapted, following transmission, to act as a receiver of acoustic signals 108A which may be reflected back from interface medium 111 due to inadequate acoustic contact. For convenience hereinafter, acoustic signal 108A may also be referred to as “reflected signal”.

Interface medium 111, which may be for example, a polyethylene membrane, is adapted to abut with the treatment region in order to substantially provide adequate acoustic contact between transducer unit 101 and the treatment region. Adequate acoustic contact generally allows for sufficient beam energy radiated by transducer element 102 to reach a target area so as to provide therapeutic results. Interface medium 111 is further adapted to reflect all of, or a portion of, pulse 108, if there is inadequate acoustic contact between transducer unit 101 and the treatment region.

Measurement system 100 is further adapted to compare an intensity (for example, energy, power), amplitude and/or other comparative parameters of reflected signal 108A to a predetermined threshold. If the intensity, amplitude and/or other comparative parameters exceeds the threshold, an indication is generally provided by measurement system 100 indicative of there being inadequate acoustic contact between transducer unit 101 and the treatment region.

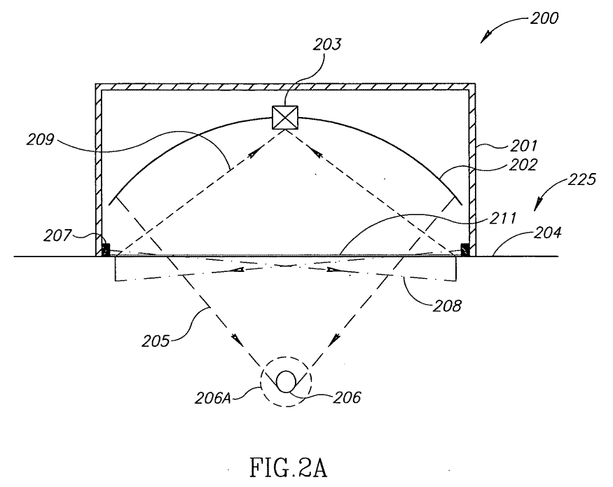

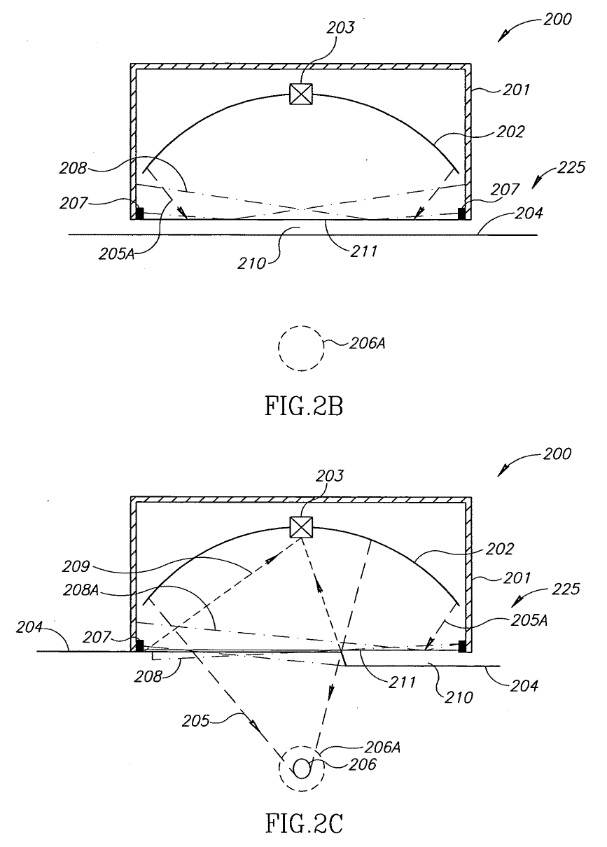

Reference is made to FIG. 2A, which schematically illustrates an exemplary TFED system 200 comprising a transducer unit 201 in a position of acoustic contact with an optical contact material such as a skin surface 204 of a patient 225, in accordance with an embodiment. Reference is also made to FIGS. 2B and 2C which schematically illustrate transducer unit 201 in a position of no acoustic contact (or inadequate acoustic contact) with skin surface 204, and in a position of partial acoustic contact (acoustic contact is inadequate in some areas) with skin surface 204, respectively, in accordance with an embodiment. Transducer unit 201 comprises a transducer element 202, a detector 203, radiating element 207, and an interface medium 211. Transducer 201 is adapted to spatially cover interface medium 211.

Transducer element 202 is adapted to radiate a focused energy beam 205 to a focal point 206 in a target area 206A. Optionally, the focused energy may be ultrasound energy. Transducer element 202 may comprise one or more transducer elements adapted to convert electrical signals (electrical energy) into ultrasound. Focusing of the ultrasound may be achieved by a geometrical design of transducer element 202, and/or by electrical characteristics of the electrical signals.

In accordance with an embodiment, radiating elements 207, which may comprise one or more radiating elements, are adapted to emit EM radiation 208 which impinge, at relatively low angles of incidence greater than the critical angle, for example, greater than 41.8 degrees as between air and the contact material, on a substantially major portion of interface medium 211. Optionally, the critical angle may be greater or less than 41.8 degrees, depending on the type of interface medium 211 and/or contact material. In some embodiments, radiating elements 207 are positioned at one or more locations in relatively close proximity to a perimeter of interface medium 211. EM radiation 208 may comprise visible light, IR light including NIR, UV light, laser, or any other type of EM radiation adapted to be totally, or optionally partially reflected by interface medium 211 when in contact with a non-optical contact material such as, for example, air 210. EM radiation 208 may be further adapted to illuminate, and/or optionally heat, optical contact materials such as, for example, skin surface 204 when interface medium 211 is in contact with the optical contact material. Radiating elements 207 may comprise sources of EM wave emissions such as, for example, light emitting diodes (LED), including visible, infrared (including NIR), and/or ultraviolet LED; and/or laser diodes; or any combination thereof.

In accordance with an embodiment, interface medium 211 comprises a relatively high index of refraction compared to non-optical contact materials, and is adapted to substantially totally reflect EM radiation 208 when in contact with the non-optical contact materials. For example, in FIG. 2B, interface medium 211 may be, but not limited to, a polyethylene membrane with an index of refraction of approximately 1.5, and the non-optical contact material may be air 210 with an index of refraction of approximately 1, such that all EM radiation 208 are substantially reflected by the membrane (none of the EM radiation reach skin surface 204). Furthermore, interface medium 211 is adapted to substantially allow EM radiation 208 to pass through into optical contact materials such as skin and/or oil, which have indices of refraction substantially similar to that of the medium. For example, in FIG. 2A, interface medium 211 may be, but not limited to, the polyethylene membrane and the optical contact material may be skin surface 204, such that substantially all EM radiation 208 pass through the membrane into the skin surface. Optionally, interface medium 211 may be adapted to allow EM radiation 208 to pass through portions of the interface medium in contact with optical contact materials, and to reflect the EM radiation in those portions of the interface medium in contact with non-optical contact materials. For example, in FIG. 2C, EM radiation 208 impinging on a portion of the polyethylene membrane in contact with skin surface 204 pass through the portion of the membrane into the skin surface, while the EM radiation impinging on those portions of the membrane in contact with air 210 are reflected by the membrane.

In accordance with an embodiment, detector 203 is adapted to detect EM radiation 208 which pass through interface medium 211 and are reflected by the optical contact material into a field of view (fov) of the detector. The detected EM radiation 209, for example light, reflected from skin surface 204 are acquired by detector 203, the detector adapted to reproduce an image, and/or a map, of the treatment region wherein are shown areas of acoustic contact. Optionally, areas of inadequate acoustic contact are shown. Detector 203 may comprise, for example, an optical imager adapted to capture light from a laser source and/or from an IR source, including an NIR source. Optionally, detector 203 may comprise a camera such as a CCD (charge coupled device) or a CMOS (complementary metal-oxide semiconductor) camera. In some embodiments, detector 203 comprises a camera with a fov adapted to substantially cover the whole area of polyethylene membrane 211. Images reproduced by detector 203 may show areas in the treatment region where there is no acoustic contact as relatively dark areas. This is due to inadequate illumination of skin 204 (EM radiation 208 do not reach areas of inadequate acoustic contact and are not reflected back as detected EM radiation 209). Furthermore, areas where there is acoustic contact are shown in the images as illuminated (EM radiation 208 reach areas of acoustic contact and are reflected back as detected EM radiation 209).

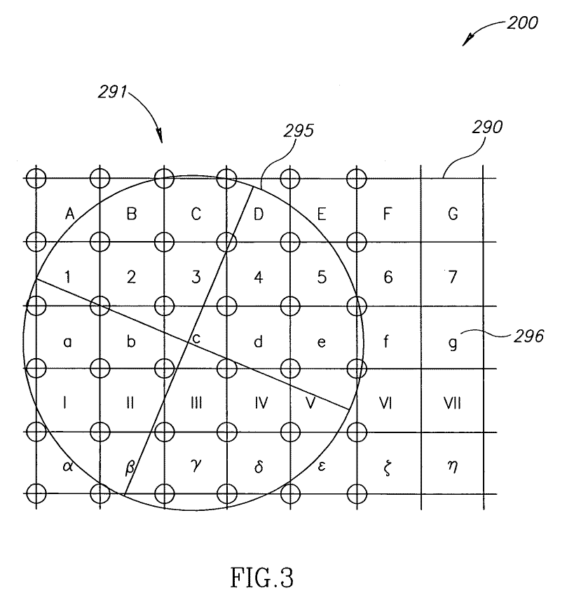

Reference is also made to FIG. 3, which schematically shows an exemplary treatment area tracking grid 290, and a visual indicator such as a crosshair 295, comprised in interface medium 211, positioned over the tracking grid, as viewed from detector 203, in accordance with an embodiment. Tracking grid 290 is adapted to divide a treatment area 291 into traceable treatment regions 296, the treatment regions comprising squares identified by a roman numeral or a letter. Optionally, treatment regions 296 may comprise other shapes such as circular, triangular, rectangular, or any other polygonal shape, or any combination thereof. Optionally, treatment regions 296 may be identified by symbols, colors, and/or any type of markings suitable for identifying the treatment regions such that one region may be distinguished from a second region. Tracking grid 290 may be painted (or drawn) unto skin 204 over treatment area 291 with visible ink, or optionally invisible ink which is visible only when exposed to EM radiation, for example, light. Optionally, tracking grid 290 may be preformed and adhered to skin 204 over treatment area 291. Optionally, tracking grid 290 may be prepared on skin 204, over treatment area 290, using an adhesive material adapted to transfer the TFED without causing substantial interference. Optionally, tracking grid 290 may comprise a gel pad which is placed over treatment area 291 outlining each treatment region 296 in treatment area 291. Optionally, treatment regions 296 are adapted to provide visual feedback to the operator according to an amount of energy delivered to the region; for example, a color of the region may change, or markings are changed or disappear, and the like. Additionally or alternatively, the visual feedback is provided when the energy delivered has reached a predetermined maximum amount.

In some embodiments, tracking grid 290 may comprise a matrix code (bar code) (not shown) wherein each treatment region 296 is identified by a bar code adapted to be read by detector 203. The bar code is decoded by detector 203 and compared in system 200 with preprogrammed information in order to determine the position of transducer 201. Detector 203 may include a bar code reader, which may comprise an electromagnetic scanner such as, for example, a photodiode/light scanner, a laser scanner, an LED scanner, a 2 dimensional (2D) imaging scanner, and the like.

Crosshair 295 is adapted to correlate a position of transducer unit 201 with respect to a particular treatment region 296 in treatment area 291. Detector 203 is adapted to acquire an image of crosshair 295 over tracking grid 290, the image adapted to provide a system operator with a substantially exact location of transducer unit 201 inside treatment area 291; that is, in which treatment region 296 transducer unit 201 is located. Optionally, the image may change as the operator manually moves transducer unit 201, including crosshair 295, over tracking grid. Optionally, a robot arm may move transducer unit 201 over the tracking grid.

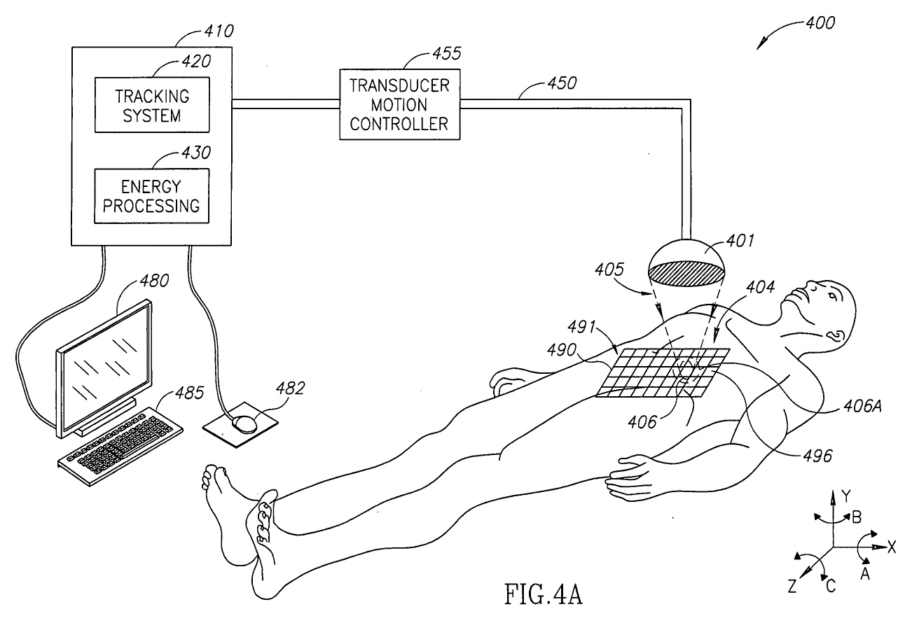

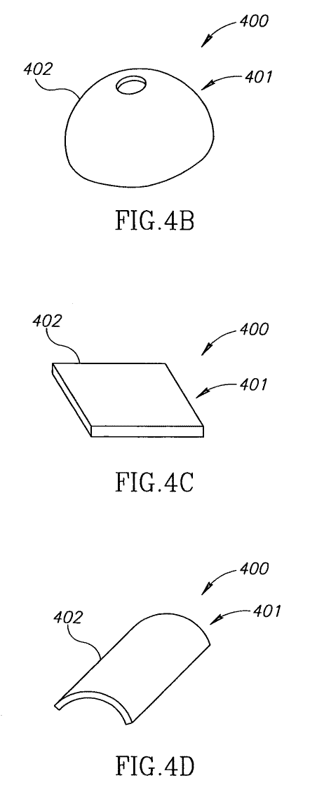

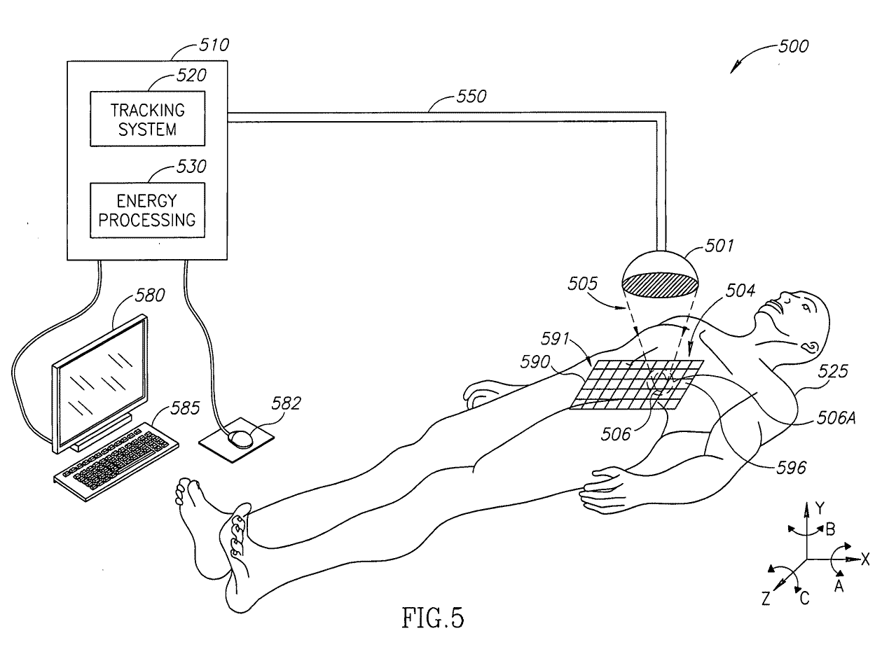

Reference is made to FIG. 4A, which schematically shows an exemplary system for delivering therapeutic focused energy 400, comprising an automatic transducer-positioning element 450, for example, a robot arm, in accordance with an embodiment. System 400 comprises a base unit 410 which includes an energy processor 430 and a tracking system 420; a transducer unit 401 which connects to base unit 410 through robot arm 450; a transducer motion controller 455; a display 480; and data input means such as a keyboard 485 and a mouse 482. TFED system 400, including transducer unit 401 may be the same or substantially similar to that shown in FIGS. 2A-2C at 200 and 201, respectively. Reference is also made to FIGS. 4B-4D which schematically show isometric views of exemplary transducer elements 402, in accordance with some embodiments.

In accordance with an embodiment, transducer unit 401 comprises transducer element 402 adapted to convert electrical signals (electrical energy) received from energy processor 430 into focused energy, for example ultrasound, and to radiate a TFED beam 405 to a patient 425. In some embodiment, transducer element 402 may be dome-shaped as shown in FIG. 4B. Optionally, transducer element 402 may be shaped as a flat plate, as shown in FIG. 4C. Optionally, transducer element 402 may be an elongated, convex-shaped plate, as shown in FIG. 4D. Optionally, transducer element 402 may comprise any shape adapted to focus the focused energy to a focal point in a target area, by direct focusing or optionally, by the use of lenses and/or other radiation focusing devices.

Transducer unit 401 is further adapted to reproduce an image, which may include a map, of treatment region 496, located within a treatment area 491, showing areas of acoustic contact. Optionally, areas of inadequate acoustic contact are shown. Optionally, transducer unit 401 is further adapted to activate a self-test to check for air bubbles in the oil-type fluid comprised in the transducer unit. Optionally, patient 425 comprises a tracking grid 490 painted (or drawn) on patient's skin 404, or optionally adhered to the skin, which combined with an optional crosshair (not shown) on transducer unit 401, are adapted to provide a system operator with a substantially exact location of the transducer unit 401 on treatment area 491. TFED beam 405, patient 425, treatment region 496, treatment area 491, tracking grid 490, skin 404, and the crosshair, may be the same or substantially similar to that shown in FIGS. 2A-2C and FIG. 3 at 205, 225, 296, 291, 290, 204, and 295. TFED beam 405 converges at a focal point 406 in a target area 406A below a treatment region 496, the focal point substantially located at the center of the crosshair. Optionally, transducer unit 401 may comprise a rolling element (not shown), for example a roller, adapted to roll over tracking grid 490 when the transducer unit is moved from treatment region 496 to treatment region. Movement of the roller is accompanied by system 400 providing a visual and/or aural feedback, the feedback adapted to alert the operator of transducer unit 401 movement following delivery of focused energy in treatment region 491.

Energy processor 430 is adapted to send electrical energy to transducer unit 401. Additionally, processor 430 is adapted to cool transducer unit 401, and additional components as may be required to ensure effective TFED transmission to focal point 406 in target area 406A. Processor 430 is further adapted to trigger transducer unit 401. Optionally, energy processor 430 is adapted to activate a self-test in transducer unit 401 to check for air bubbles in the oil-type fluid comprised in the transducer unit.

In accordance with an embodiment, tracking system 420 is adapted to process the images acquired by transducer 401 and generate tracking images of a position of transducer 401 in treatment area 491. The position of transducer unit 401, which corresponds with a position of focal point 406 within treatment region 496, may be determined by displaying, on display 480, an image of the position of the crosshair relative to identified treatment regions 496 in tracking grid 490. Furthermore, the images may show areas of adequate acoustic contact and/or inadequate acoustic contact. Optionally, the images may show treatment regions 496 in tracking grid 490 which have received a predetermined maximum amount of energy. These treatment regions 496 may be identified in tracking grid 490 by a change of color, size and/or shape in the treatment region, or by an appearance of markings and/or symbols, or optionally disappearance of markings and/or symbols, or any combination thereof.

In accordance with an embodiment, responsive to the tracking images displayed showing transducer 401 position and/or transducer acoustic contact, the operator may input tracking signals which are sent to transducer motion controller 455. The tracking signals may comprise control signals associated with moving transducer 401 in order to correctly position transducer 401 within a treatment region 496, as may be required for proper focusing of focal point 406 within target region 406A, and/or to obtain adequate acoustic contact. Moving the transducer 401 may be done in a continuous manner, at a constant speed or optionally, at a variable speed. Optionally, moving the transducer 401 may be performed intermittently, according to a predetermined time criteria. Moving of transducer 401 may comprise transducer displacement with 1, 2, 3, 4, 5, or 6 degrees of freedom. Displacement may be along an x-axis, y-axis, and/or z-axis; tilting by partial or full rotation around the x-axis as shown by A (roll), y-axis as shown by B (yaw), and/or z-axis as shown by C (pitch); or any combination thereof. Optionally, the tracking signals may additionally comprise control signals associated with moving transducer 401 from one treatment region 496 to a second treatment region in treatment area 491.

In accordance with an embodiment, transducer motion controller 455, responsive to tracking signals received, is adapted to shift robot arm 450 finite distances along the x, y, and/or z axes, and optionally roll, yaw and/or pitch, or any combination thereof. Optionally, transducer motion controller 455 may be further adapted to allow transducer unit 401 to translate on robot arm 450 with 1, 2, 3, 4, 5, or 6 degrees of freedom. Optionally, transducer motion controller 455 is adapted to move robot arm 450 from one treatment region 496 to a second target region, and to correctly position transducer unit 401 over the treatment region. Transducer motion controller 455 may comprise electric motors, such as for example, stepper motors, to effect the movement in robot arm 450, and optionally transducer unit 401. Optionally, in some embodiments, hydraulic, pneumatic, and/or magnetic means, or any combination thereof, may be used to effect movement in robot arm 450, and optionally transducer unit 401. Optionally, electric motors may be used in combination with the hydraulic, pneumatic, and/or magnetic means, or any combination thereof. Optionally, movement of robot arm 450 may be operator controlled. Additionally or alternatively, robot arm 450 movement is automatic, guided by comparing the processed images with pre-stored information of tracking grid 490. Automatic movement of robot arm 450 may follow a raster pattern, or optionally a non-raster pattern. Optionally, automatic movement may be top-bottom, or left-right, clockwise, or counter-clockwise, or any combination thereof.

In accordance with an embodiment, robot arm 450 is responsive to control signals from motion controller 455. Robot arm 450 is adapted to move transducer unit 401 finite distances along the x, y, and/or z axes, and optionally roll, yaw and/or pitch, or any combination thereof. Optionally, robot arm may be further adapted to allow transducer unit 401 to translate on the robot arm with 1, 2, 3, 4, 5, or 6 degrees of freedom. Robot arm 450 may further be adapted to move transducer unit 401 inside a treatment region 496, and to correctly position the transducer inside the treatment region. In addition, robot arm 450 may be adapted to move transducer 401 from one treatment region 496 to another treatment region in tracking grid 490.

Display 480, keyboard 485 and mouse 482 are adapted to allow the system operator input/output data interface with system 400. Information may be displayed in display 480 such as, for example, position of transducer 401 in tracking grid 490; transducer displacement; transducer 401 position relative to a next treatment region 496; close up images of treatment area 491 and/or treatment region 496; among numerous others. Input data through keyboard 485 and/or mouse 482 may also be displayed in display 480, such as, for example, a predetermined time criteria for intermittent movements, rate of movement (constant speed or variable), type of movement (x, y and/or z—axis translation, and/or roll, yaw, pitch, or any combination thereof), among numerous other.

Reference is made to FIG. 5, which schematically shows an exemplary system for delivering therapeutic focused energy 500, comprising a manual transducer-positioning element 550, for example a mechanical arm, in accordance with an embodiment. System 500 comprises a base unit 510 which includes an energy processor 530 and a tracking system 520; a transducer unit 501 which connects to base unit 510 through mechanical arm 550; a display 580; and data input means such as a keyboard 585 and a mouse 582. Base unit 510, energy processor 530, tracking system 520, transducer unit 501, display 580, keyboard 585 and mouse 582 are the same or substantially similar to that shown in FIG. 4A at 410, 430, 420, 401, 480, 485 and 482. Comprised in transducer unit 510 may be a transducer element (not shown) the same or substantially similar to that shown in FIG. 4B at 402. Optionally, the transducer element may be the same or substantially similar to that shown in FIG. 4C at 402. Optionally, the transducer element may be the same or substantially similar to that shown in FIG. 4D at 402. TFED beam 505, patient 525, treatment region 596, treatment area 591, tracking grid 590, skin 504, and the crosshair, may be the same or substantially similar to that shown in FIGS. 2A-2C and FIG. 3 at 205, 225, 296, 291, 290, 204, and 295. TFED beam 505 converges at a focal point 506 in a target area 506A below a treatment region 596, the focal point substantially located at the center of the crosshair.

In accordance with an embodiment, mechanical arm 550 is adapted to allow a system operator to manually move transducer unit 501 finite distances along the x, y, and/or z axes, and optionally roll, yaw and/or pitch, or any combination thereof. Optionally, mechanical arm 550 may be further adapted to allow transducer unit 501 to manually translate on the mechanical arm with 1, 2, 3, 4, 5, or 6 degrees of freedom. Mechanical arm 550 may further be adapted to allow manual movement of transducer unit 501 from one treatment region 596 to a second treatment region in tracking grid 590, and to manually position the transducer in the treatment region.

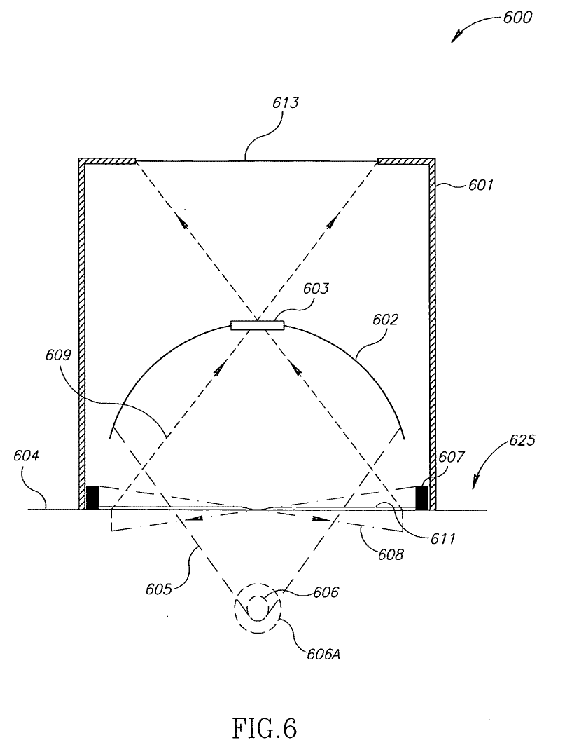

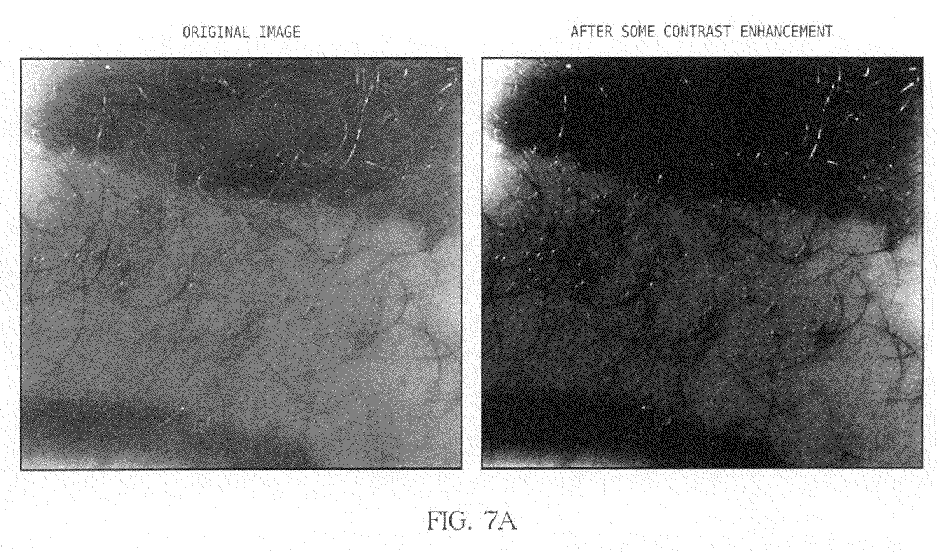

Reference is made to FIG. 6, which schematically illustrates an exemplary TFED system 600 comprising a transducer unit 601 in a position of acoustic contact with an optical contact material such as a skin surface 604 of a patient 625, in accordance with another embodiment. Transducer unit 601 comprises a transducer element 602, an imaging lens 603, an imaging (optical) screen 613, a radiating element 607, and an interface medium 611. Transducer 601 is adapted to spatially cover interface medium 611.

Transducer element 602, which may be the same or substantially similar to that shown in FIGS. 2A-2C at 202, or optionally FIGS. 4B-4D at 402, is adapted to radiate a focused energy beam 605 to a focal point 606 in a target area 606A. In accordance with another embodiment, radiating elements 607, which may comprise one or more radiating elements, are adapted to emit EM radiation 608 which impinge, at relatively low angles of incidence greater than the critical angle, for example, greater than 41.8 degrees in case of air (refractive index=1) and Lucite (refractive index=1.5), on a substantially major portion of interface medium 611. Optionally, the critical angle may be greater or lesser than 41.8 degrees, depending on the type of interface medium 611 and/or contact material. In some embodiments, radiating elements 607 are positioned at one or more locations in relatively close proximity to a perimeter of interface medium 611. Radiating elements 607 and interface medium 611 may be the same or substantially similar to that shown in FIGS. 2A-2C at 207 and 211.

In accordance with another embodiment, EM radiation 608 passing through interface medium 611, in areas of acoustic contact, are reflected by an optical contact material through lens 603 towards imaging screen 613. The reflected EM radiation may be hereinafter referred to as reflected EM radiation 609, Imaging screen 613, which may be located on an opposite side of lens 603 from that of interface medium 611, is adapted to capture reflected EM radiation 609 and reconstruct an image of areas where there is acoustic contact (illuminated areas). Optionally, imaging screen 613 may also reconstruct an image of areas where there is no acoustic contact (may be shown as dark areas in the imaging screen). Optionally, imaging screen 613 may be a liquid crystal display (LCD). Optionally, mirrors (not shown) may be used to direct the image of the illuminated areas to a remote imaging screen (not shown).

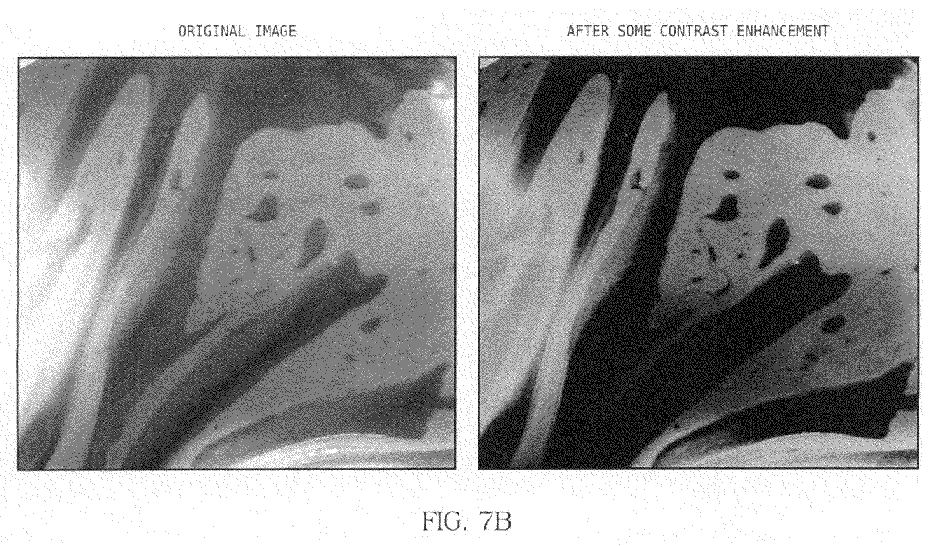

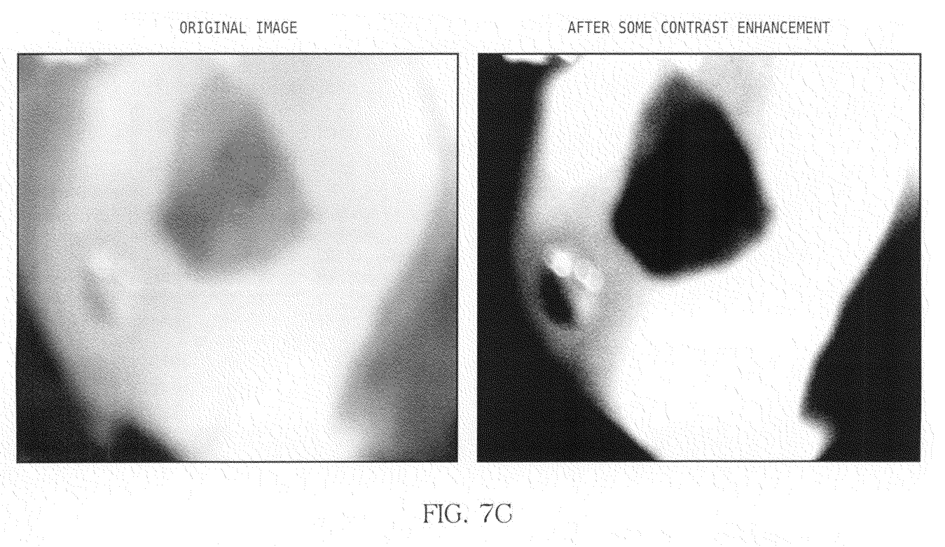

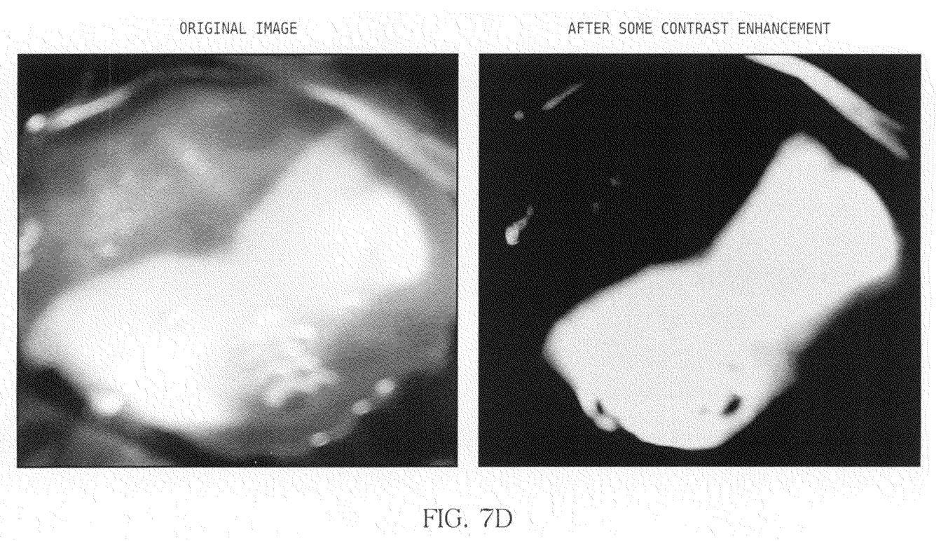

Reference is made to FIGS. 7A-7D which show exemplary images of different optical contact materials, as acquired by a TFED system comprising a transducer unit comprising three EM radiating elements (LEDs), in accordance with an embodiment. The LEDs were position equidistant from one another along a circumference of the transducer unit, in close proximity to a polyethylene membrane serving as an interface medium. Areas of acoustic contact are shown in the images as illuminated, while areas of inadequate acoustic contact are shown in dark. FIG. 7A includes an original image of a sample skin area as acquired by the detector (a consumer digital CCD camera) and the same image after contrast enhancement. FIG. 7B includes an original image of a sample rubber as acquired by the detector and the same image after contrast enhancement. FIG. 7C includes an original image of a simulation of an air bubble in castor oil as acquired by the detector and the same image after contrast enhancement. FIG. 7D includes an original image of an example of partial acoustic contact, where the interface medium and skin are in acoustic contact in a central portion of the membrane, as acquired by the detector; and the same image after contrast enhancement.

In the description and claims of embodiments of the present invention, each of the words, “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art.

What is claimed is:

1. A transducer unit adapted to deliver ultrasonic focused energy, the transducer unit comprising:
an interface medium adapted to contact at least a portion of a treatment region in a treatment area; and
an electromagnetic (EM) radiating element adapted to transmit EM radiation towards the interface medium, wherein a reflection of the EM radiation from the interface medium is indicative of an extent of acoustic contact.

2. The transducer unit of claim 1, wherein the EM radiating element is adapted to transmit EM radiation such that the radiation impinges on the interface medium at an angle range in which total internal reflection occurs from the interface medium when in air.

3. The transducer unit of claim 1, wherein the interface medium is adapted to reflect at least a portion of the EM radiation in areas of inadequate acoustic contact within the treatment region.

4. The transducer unit of claim 1, wherein said interface medium is adapted to pass a portion of the EM radiation in areas of adequate acoustic contact with the treatment region.

5. The transducer of claim 1, wherein the EM radiation comprises visible light.

6. The transducer of claim 1, wherein the EM radiation comprises laser.

7. The transducer of claim 1, wherein the EM radiation comprises infrared (IR) radiation.

8. The transducer of claim 1, wherein the EM radiation comprises near infrared (NIR) radiation.

9. The transducer of claim 1, wherein the EM radiation comprises ultraviolet (UV) radiation.

10. The transducer unit of claim 1, wherein the EM radiation spatially cover the interface medium.

11. The transducer unit of claim 1, wherein said EM radiating element is an LED (light emitting diode).

12. The transducer unit of claim 1, further comprising a detector adapted to acquire detected EM radiation reflected from the treatment region.

13. The transducer unit of claim 12, wherein said detector is further adapted to acquire an image of the treatment region from the detected EM radiation.

14. The transducer unit of claim 13, wherein said image comprises areas of acoustic contact.

15. The transducer unit of claim 13, wherein said image comprises areas of inadequate acoustic contact.

16. The transducer unit of claim 12, wherein said detector comprises an optical imager.

17. The transducer unit of claim 16, wherein the optical imager comprises a camera.

18. The transducer unit of claim 1, wherein said interface medium is a polyethylene membrane.

19. The transducer unit of claim 1, wherein said interface medium comprises a visual indicator adapted to show a focal point position of the focused energy.

20. The transducer unit of claim 1, further adapted for lysing adipose tissue.

21. The transducer unit of claim 1, further adapted for lysing cellulite.

22. A system for delivering ultrasonic focused energy, the system comprising:
a transducer unit for delivering the ultrasonic focused energy, the transducer unit comprising an interface medium adapted to contact at least a portion of a treatment region in a treatment area, and further comprising an electromagnetic (EM) radiating element adapted to transmit EM radiation towards the interface medium, wherein a reflection of the EM radiation from the interface medium is indicative of an extent of acoustic contact; and
an energy processing unit adapted to send electrical energy to the transducer unit.

23. The system of claim 22, wherein the transducer unit further comprises a detector adapted to acquire images of the treatment region.

24. The system of claim 23, wherein said detector is further adapted to acquire an image of the treatment region from the detected EM radiation.

25. The system of claim 24, wherein said image comprises areas of acoustic contact.

26. The system of claim 24, wherein said image comprises areas of inadequate acoustic contact.

27. The system of claim 23, wherein said detector comprises an optical imager.

28. The system of claim 27, wherein the optical imager comprises a camera.

29. The system of claim 22, further comprising a tracking system adapted to process the images acquired by the detector, and to generate tracking images of a position of the transducer unit in the treatment area.

30. The system of claim 22, wherein the transducer unit comprises a visual indicator adapted to show a focal point position of the focused energy within the treatment region.

31. The system of claim 30, wherein the transducer unit is adapted to acquire an image of the visual indicator over the treatment region.

32. The system of claim 29, further comprising a tracking grid dividing the treatment area into a plurality of treatment regions.

33. The system of claim 32, wherein the tracking grid is painted or drawn on a skin of a patient.

34. The system of claim 32, wherein the tracking grid is painted or drawn on a gel layer deposited on a skin of a patient.

35. The system of claim 32, wherein said tracking grid is adhered to a skin of a patient.

36. The system of claim 32, wherein the tracking grid comprises a matrix code adapted to be read by a bar code reader.

37. The system of claim 32, wherein the crosshair is adapted to correlate the position of the transducer unit with a treatment region in the target grid.

38. The system of claim 32, wherein the tracking grid is adapted to provide visual feedback related to a treatment region which has received a predetermined amount of focused energy.

39. The system of claim 38, wherein the visual feedback is a change of color in the treatment region.

40. The system of claim 38, wherein the visual feedback is an appearance of a marking and/or a symbol in the treatment region.

41. The system of claim 38, wherein the visual feedback is a disappearance of a marking and/or a symbol from the treatment region.

42. A method for delivering ultrasonic focused energy, the method comprising:
establishing contact between an interface medium comprised in a transducer unit for delivering the ultrasonic focused energy and at least a portion of a treatment region in a treatment area;
transmitting EM radiation towards the interface medium, wherein a reflection of the EM radiation from the interface medium is indicative of an extent of acoustic contact; and
transmitting electrical energy to the transducer unit.

43. The method of claim 42, further comprising acquiring images of the treatment region.

44. The method of claim 43, further comprising processing the images and generating tracking images of a position of the transducer unit in the treatment area.

45. The method of claim 42, further comprising showing a focal point position of the focused energy within the treatment region using a visual indicator comprised in the transducer unit.

46. The method of claim 45, further comprising acquiring an image of the visual indicator over the treatment region.

47. The method of claim 44, further comprising dividing the treatment area into a plurality of treatment regions within a tracking grid.

48. The method of claim 44, further comprising painting the tracking grid on a skin of a patient.

49. The method of claim 44, further comprising adhering the tracking grid to a skin of a patient.

50. The method of claim 44, further comprising correlating the position of the transducer unit with a treatment region in the tracking grid using the visual indicator.

51. The method of claim 47, further comprising providing provide visual feedback in the tracking grid related to a treatment region which has received a predetermined amount of focused energy.

52. The method of claim 51, wherein the visual feedback is a change of color in the treatment region.

53. The method of claim 51, wherein the visual feedback is an appearance of a marking and/or a symbol in the treatment region.

54. The method of claim 51, wherein the visual feedback is a disappearance of a marking and/or a symbol from the treatment region.
